(19) United States
(12) Patent Application Publication
(10) Pub. No.: US 2017/0001944 A1
(43) Pub. Date: Jan. 5, 2017

(54) CHEMICAL PROCESS TO CONVERT MUCIC ACID TO ADIPIC ACID

(71) Applicant: AGENCY FOR SCIENCE TECHNOLOGY AND RESEARCH, Connexis, Singapore (SG)

(72) Inventors: Yugen ZHANG, Singapore (SG); Xiukai Li, Singapore (SG)

(21) Appl. No.: 15/102,264

(22) PCT Filed: Dec. 4, 2014
(86) PCT No.: PCT/SG2014/000575

§ 371 Date: Jun. 6, 2016

(30) Foreign Application Priority Data
Dec. 4, 2013 (SG) 201308992-5

(51) Int. Cl. C07C 69/44; C07C 67/08; C07C 51/377

(52) U.S. Cl. CPC C07C 69/44; C07C 51/377; C07C 67/08

(57) ABSTRACT

The present invention provides a method of synthesizing an ester of a saturated carboxylic acid from a saturated polyhydroxycarboxylic acid by performing a deoxydehydration reaction and a hydrogen transfer reaction.

### TECHNICAL FIELD

The present invention generally relates to a method of synthesizing an ester of a saturated polycarboxylic acid.

### BACKGROUND

The production of bulk chemicals and fuels from renewable bio-based feedstock is of significant importance for the sustainability of human society. Adipic acid (hexanedioic acid), as one of the most demanded “drop in” chemicals from bioresource, is used primarily for the large volume production of nylon-6,6 polyamide.¹ The global demand for adipic acid is growing at 3-3.5% annually and is expected to reach 3.3 million metric tons in 2016. Currently, the commercial adipic acid is mainly derived from the petroleum-based cyclohexane, through which process a nitric acid oxidation is involved. Besides the non-renewable feedstock cyclohexane source used in this synthetic route, the emission of large amounts of nitrous oxides (N₂O, NO, and NO₂) during the oxidation process is also a significant environmental concern. It is therefore highly desirable to develop a sustainable and environmentally friendly process for the production of adipic acid from renewable feedstock.

To produce adipic acid from renewable precursors, combined biocatalytic and chemocatalytic pathways have therefore been created with cis,cis-muconic acid as the key intermediate. Muconic acid, as well as its various derivatives are also popular chemical intermediates for the production of fibers and plastics. In the reported conversions of glucose to muconic acid with multiple-step fermentation processes, the biocatalytic production of muconic acid requires the assists of several different kinds of enzymes and the product yield and efficiency are very low. As a comparison, the conversion of muconic acid to adipic acid is limited in being a rather straight forward hydrogenation reaction. Adipic acid preparations via chemocatalytic hydrogenations of furan-2,5-dicarboxylic acid (FDCA) and glucaric acid have been reported. However, drastic reaction conditions such as the strong halogen acid and the high pressure (more than 50 bar) of H₂ were employed. The step by step hydrogenolysis of 5-hydroxymethylfurfural (HMF) can produce 1,6-hexanediol (a potential precursor to adipic acid) with high selectivity albeit low HMF conversion. The harsh reaction conditions and low efficiency of these methods make them unlikely to be industrialized.

The key challenge for the conversion of highly oxygen-rich bio-compounds (sugar, sugar acids and sugar alcohols) to industrial bulk chemicals is to develop highly efficient deoxygenation catalytic systems, which can selectively convert bio-resources to target chemicals. Recently, a deoxydehydration (DODH) reaction was successfully applied in the conversion of polyols including sugar alcohols to conjugated alkenes. Although the DODH reaction conditions are rather mild, the choice of substrate was limited to polyols and the reaction selectivity was not sufficiently high.

There is therefore a need to provide a method for synthesizing an ester of a saturated polycarboxylic acid, such as an ester of adipic acid, which ameliorates one or more of the disadvantages described above.

### SUMMARY

In a first aspect, there is provided a method for synthesizing an ester of a saturated polycarboxylic acid, the method comprising the steps of: (a) subjecting a saturated polyhydroxycarboxylic acid to a deoxydehydration catalyst to remove hydroxyl groups; and (b) performing a hydrogen transfer reaction to form the ester of a saturated polycarboxylic acid.

Advantageously, the disclosed method allows a highly efficient conversion of the saturated polyhydroxycarboxylic acid to the saturated polycarboxylic acid via the deoxydehydration (DODH) reaction catalyzed by the deoxydehydration catalyst. Excellent, almost quantitative yield (99%) may be achieved for the DODH reaction compared to yields of approximately 60% obtainable using conventional methods. By combining DODH with a hydrogen transfer reaction, a polyhydroxycarboxylic acid is successfully converted to a saturated polycarboxylic in excellent yield. Further advantageously, the reaction proceeds under mild conditions. As such, the disclosed method may simplify the synthetic process of saturated carboxylic acids such as adipic acids from polyhydroxycarboxylic acids such as mucic acid, as the reaction conditions are milder and more time- and cost-efficient compared to conventional methods.

Further advantageously, the disclosed method demonstrates a high efficient, simple and green protocol for the production of renewable saturated polycarboxylic acid from polyhydroxycarboxylic acids such as aldaric acids, which would be useful in utilizing biofuels for generating biorenewable materials. The disclosed method has large potential in utilization in synthesis of various industrial chemicals from biofuels including various sugars.

In the disclosed method, the hydrogen transfer reaction in step (b) may be performed in the presence of a hydrogen transfer catalyst.

Advantageously, the steps (a) and (b) may be performed in a single reaction vessel (i.e. a “one-pot” method).

Optionally, in the disclosed method, step (a) and step (b) may be performed concurrently. In embodiments where step (a) and step (b) are performed concurrently, the polyhydroxycarboxylic acid may be contacted with the deoxydehydration catalyst in the presence of a hydrogen transfer catalyst.

In another embodiment, step (a) and (b) may be performed consecutively. In the embodiment where steps (a) and (b) are performed consecutively, the deoxydehydration catalyst may produce at least one intermediate compound. In another embodiment, the at least one intermediate compound produced may be an ester of an unsaturated polycarboxylic acid after step (a). The ester of an unsaturated polycarboxylic acid may then subjected to step (b) to produce the ester of a saturated polycarboxylic acid.

Advantageously, the nature of the reaction allows the reaction to be performed both concurrently and consecutively, allowing versatility in the way the reaction may be performed. Almost quantitative yields may be achieved converting the polyhydroxycarboxylic acid to the saturated polycarboxylic acid and then to the unsaturated polycarboxylic acid, either in a two-step or one-step process.

In another embodiment, the ester of an unsaturated polycarboxylic acid may comprise esters of muconic acid. The ester of a saturated polycarboxylic acid may comprise esters of adipic acid. In another embodiment, the polyhydroxycarboxylic acid may comprise mucic acid.

In a second aspect, there is disclosed an ester of adipic acid product synthesized by the method according to the first aspect.

In a third aspect, there is provided a method for synthesizing an ester of adipic acid, the method comprising the step of subjecting mucic acid to a deoxydehydration catalyst in the presence of a hydrogen transfer catalyst to form the ester of adipic acid.

In a fourth aspect, there is provided a method for synthesizing an ester of adipic acid, the method comprising the steps of: (a) subjecting mucic acid to a deoxydehydration catalyst to form an ester of muconic acid; and (b) performing a hydrogen transfer reaction on the ester of muconic acid to form the ester of adipic acid.

In a fifth aspect, there is provided a method for synthesizing a saturated carboxylic acid, the method comprising the steps of; (a) subjecting a polyhydroxycarboxylic acid to a deoxydehydration catalyst to remove hydroxyl groups; (b) performing a hydrogen transfer reaction to form the ester of a saturated polycarboxylic acid; (c) hydrolysing the ester to form the saturated carboxylic acid.

### DEFINITIONS

The following words and terms used herein shall have the meaning indicated:

The term “concurrent”, for the purposes of the present disclosure, refers to two or more reactions occurring at the same time. The term “concurrently” should be construed accordingly.

The term “consecutive”, for the purposes of the present disclosure, refers to performing one reaction after the other, in a chronological sequence. The term “consecutively” should be construed accordingly.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the terms “about” and “approximately”, in the context of concentrations of components of the formulations, or where applicable, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Optional Embodiments

Illustrative, non-limiting embodiments of a method of synthesizing an ester of a saturated carboxylic acid according to the first aspect will now be disclosed.

A method for synthesizing an ester of a saturated polycarboxylic acid, the method comprising the steps of (a) subjecting a saturated polyhydroxycarboxylic acid to a deoxydehydration catalyst to remove hydroxyl groups; and (b) performing a hydrogen transfer reaction to form the ester of a saturated polycarboxylic acid is described.

The hydrogen transfer reaction in step (b) may be performed in the presence of a hydrogen transfer catalyst.

The steps (a) and (b) may be performed in a single reaction vessel (i.e. a “one-pot” method).

Step (a) and step (b) may be performed concurrently. In embodiments where step (a) and step (b) are performed concurrently, the polyhydroxycarboxylic acid may be contacted with the deoxydehydration catalyst in the presence of a hydrogen transfer catalyst.

In embodiments where step (a) and step (b) are performed concurrently, the polyhydroxycarboxylic acid, deoxydehydration catalyst and hydrogen transfer catalyst may together form a “reaction mixture”, to produce the ester of the saturated polycarboxylic acid.

Step (a) and (b) may be performed consecutively. In the embodiments where steps (a) and (b) are performed consecutively, the deoxydehydration catalyst may produce at least one intermediate compound. The at least one intermediate compound produced may be an ester of an unsaturated polycarboxylic acid after step (a). The ester of an unsaturated polycarboxylic acid may then subjected to step (b) to produce the ester of a saturated polycarboxylic acid.

The polycarboxylic acid may be an unbranched-chain dicarboxylic acid containing two terminal COOH groups. The unbranched chain portion of the saturated polycarboxylic acid may be composed entirely of single bonds and may be saturated with hydrogen. The ester of an unsaturated polycarboxylic acid may be a monoester of diester of a C2 to C10 polycarboxylic acid. The ester of an unsaturated polycarboxylic acid may comprise esters of maleic acid, fumaric acid, glutanoic acid, galacturonic acid, traumatic acid or muconic acid.

In another embodiment, the ester of an unsaturated polycarboxylic acid may comprise monoester or diesters of muconic acid. Muconic acid is (2E,4E)-Hexa-2,4-dienedioic acid.

In yet another embodiment, the ester of a muconic acid may comprise propyl muconate, dipropyl muconate, butyl muconate, dibutyl muconate, pentyl muconate, dipentyl muconate, octyl muconate, dioctyl muconate or any mixture thereof. The ester of muconic acid may comprise prop-2-yl muconate, diprop-2-yl muconate, but-1-yl muconate, dibut-1-yl muconate, pent-3-yl muconate, dipent-3-yl muconate, oct-3-yl muconate, dioct-3-yl muconate or any mixture thereof.

The polycarboxylic acid may be an unbranched-chain dicarboxylic acid containing two terminal COON groups. The unbranched chain portion of the unsaturated polycarboxylic acid may have one or more double or triple bonds between the carbon atoms. The ester of a saturated polycarboxylic acid may comprise monoester or diesters of a C2 to C10 saturated polycarboxylic acid. The ester of a saturated polycarboxylic acid may comprise monoester or diesters of oxalic acid, malonic acid, succinic acid, glutaric acid, adipic acid, pimelic acid, suberic acid, azelaic acid, 5-oxopentanoic acid or sebacic acid.

The ester of a saturated polycarboxylic acid may comprise monoester or diesters of adipic acid.

In the disclosed method, the esters of adipic acid may comprise adipic acid monopropyl ester, adipic acid dipropyl ester, adipic acid monobutyl ester, adipic acid dibutyl ester, adipic acid monopentyl ester, adipic acid dipentyl ester, adipic acid monooctyl ester, adipic acid dioctyl ester or any mixture thereof. In yet another embodiment, the esters of adipic acid comprises adipic acid monoprop-3-yl ester, adipic acid diprop-3-yl ester, adipic acid monobut-1-yl ester, adipic acid dibut-1-yl ester, adipic acid monopent-3-yl etser, adipic acid dipen-3-yl ester, adipic acid monocot-3-yl ester, adipic acid dioct-3-yl ester or any mixture thereof.

The polyhydroxycarboxylic acid may have the formula HOOC—(CHOH)ₙ—COOH. N may be any integer between 1 and 10. The polyhydroxycarboxylic acid may be a C2 to C10 polyhydroxycarboxylic acid. The polyhydroxycarboxylic acid may be an aldaric acid. The aldaric acid may be D-glutaric acid, L-gularic acid, D-galactaric acid, galacturonic acid or L-galactaric acid. The polyhydroxycarboxylic acid may be mucic acid. Mucic acid may also be referred to as galactaric acid or meso-galactaric acid.

Deoxydehydration may be a reaction that simultaneously removes oxygen and hydrogen from a compound. Deoxydehydration may facilitate complete or partial dehydroxylation of a compound. The deoxydehydration reaction may remove hydroxyl groups from a compound. In the disclosed method, the deoxydehydration catalyst in step (a) comprises a rhenium catalyst or a rhenium catalyst with a co-catalyst. The rhenium catalyst may comprise rhenium acid, methyltrioxorhenium or rhenium(VII) oxide. In yet another embodiment, the co-catalyst may be a proton type liquid or solid acid. The co-catalyst may be a BrØnsted acid. The BrØnsted acid may comprise para-toluene sulfonic acid, sulfuric acid or any mixture thereof.

The hydrogen transfer reaction may be the addition of hydrogen (H₂) to a molecule from a source other than gaseous H₂. The reaction may be mediated by a catalyst.

The hydrogen transfer catalyst may be a metal-on-carbon catalyst with the metal being selected from the group consisting of platinum, palladium, ruthenium and any mixture thereof. The hydrogen transfer catalyst may comprise up to 10%, 9%, 8%, 7%, 6%, 5%, 4%, 3%, 2% or 1% of the reaction mixture. The hydrogen transfer catalyst may comprise up to 5 mol% of the reaction mixture. Optionally, the said hydrogen transfer catalyst may be selected from the group consisting of 5 mol % Ru/C, 5 mol % Pd/C, 5 mol % Pt/C and any mixture thereof.

In embodiments where step (a) and step (b) are performed concurrently, the said method of synthesizing the ester of a saturated polycarboxylic acid may comprise the use of an alcohol solvent. The alcohol solvent may be selected from the group consisting of propanol, butanol, pentanol, hexanol, heptanol, octanol and any mixture thereof. The alochol solvent may be 2-propanol, 1-butanol, 3-pentanol, 3-octanol or any mixture thereof. In embodiments where step (a) and step (b) are performed concurrently, the said method of synthesizing the ester of a saturated polycarboxylic acid may be 3-pentanol.

In embodiments where step (a) and step (b) are performed concurrently, the synthesis pathway may be performed at a temperature in the range of about 120° C. to about 200° C., about 120° C. to about 140° C., 120° C. to about 160° C., 120° C. to about 180° C., about 140° C. to about 160° C., about 140° C. to about 180° C., about 140° C. to about 200° C., about 160° C. to about 180° C., or about 160° C. to about 200° C.

In embodiments where step (a) and step (b) are performed concurrently, the synthesis pathway may performed for a period of about 24 hours to about 36 hours, about 24 hours to 30 hours or about 30 hours to about 36 hours.

In embodiments where step (a) and (b) are performed consecutively, different solvents may be used in step (a) and step (b).

In embodiments where step (a) and step (b) are performed consecutively, the deoxydehydration reaction in step (a) may comprise the use of an alcohol solvent. The alcohol solvent in step (a) may be selected from the group consisting of propanol, butanol, pentanol, hexanol, heptanol, octanol and any mixture thereof. The alcohol solvent in step (a) may be 2-propanol, 1-butanol, 3-pentanol, 3-octanol or any mixture thereof.

The alcohol solvent in step (b) may be selected from the group consisting of propanol, butanol, pentanol, hexanol, heptanol, octanol and any mixture thereof. The alcohol solvent in step (b) may be 2-propanol, 1-butanol, 3-pentanol, 3-octanol or any mixture thereof. The alcohol solvent used for the hydrogen transfer reaction in step (b) may be 3-pentanol.

In embodiments where step (a) and step (b) are performed consecutively, step (a) may be performed at a temperature in the range of about 90° C. to about 180° C., about 90° C. to about 120° C., about 90° C. to about 150° C., about 120° C. to about 150° C., about 120° C. to about 180° C. or about 150° C. to about 180° C. Step (b) may be performed at a temperature in the range of about 120° C. to about 200° C., about 120° C. to about 140° C., 120° C. to about 160° C., 120° C. to about 180° C., about 140° C. to about 160° C., about 140° C. to about 180° C., about 140° C. to about 200° C., about 160° C. to about 180° C., or about 160° C. to about 200° C.

In embodiments where step (a) and step (b) are performed consecutively, step (a) may be performed for a period of about 4 hours to about 24 hours, about 4 hours to about6 hours, about 4 hours to about 8 hours, about 4 hours to about 12 hours, about 6 hours to about 8 hours, about 6 hours to about 12 hours, about 6 hours to about 24 hours, about 8 hours to about 12 hours, about 8 hours to about 24 hours or about 12 hours to about 24 hours. The step (b) may be performed for a period of about 6 hours to about 24 hours, about 6 hours to about 8 hours, about 6 hours to about 12 hours, about 6 hours to about 24 hours, about 8 hours to about 12 hours, about 8 hours to about 24 hours or about 12 hours to about 24 hours.

An ester of adipic acid product synthesized by the method as defined above is also described.

A method for synthesizing an ester of adipic acid, the method comprising the step of subjecting mucic acid to a deoxydehydration catalyst in the presence of a hydrogen transfer catalyst to form the ester of adipic acid is also described.

A method for synthesizing an ester of adipic acid, the method comprising the steps of: (a) subjecting mucic acid to a deoxydehydration catalyst to form an ester of muconic acid; and (b) performing a hydrogen transfer reaction on the ester of muconic acid to form the ester of adipic acid is also described.

A method for synthesizing a saturated carboxylic acid, the method comprising the steps of; (a) subjecting a polyhydroxycarboxylic acid to a deoxydehydration catalyst to remove hydroxyl groups; (b) performing a hydrogen transfer reaction to form the ester of a saturated polycarboxylic acid; and (c) hydrolysing the ester to form the saturated carboxylic acid. The hydrolysis step may be performed by any method known to a person skilled in the art. The hydrolysis may be performed in the presence of aqueous acid or aqueous base. The hydrolysis may remove the ester groups from the ester of the saturated polycarboxylic acid of step (b). The hydrolysis step may remove one or both the ester groups of the ester of the saturated polycarboxylic acid of step (b).

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a reaction scheme showing the conversion of mucic acid to muconic acid and adipic acid.

FIG. 2A is a graph showing deoxydehydration (DODH) of mucic acid with methyltrioxorhenium (MTO) catalyst.

FIG. 2B is a graph showing deoxydehydration (DODH) of mucic acid with methyltrioxorhenium (MTO) and TsOH catalyst. Reaction conditions: mucic acid (1.0 mmol), catalyst (5.0 mol %), 3-pentanol (20.0 ml), 120° C.

FIG. 3 is a reaction schemes showing deoxydehydration (DODH) of mucic acid and mucic acid diester to muconates.

FIG. 4 is a graph showing the progression of DODH of diethyl mucate to muconates (5+7) with methyltrioxorhenium (MTO) catalyst. Reaction conditions: diethyl mucate (1.0 mmol), catalyst (5.0 mol %), 3-pentanol (20.0 ml), 120° C.

FIG. 5 is a reaction scheme showing the transfer-hydrogenation of trans, trans-muconic acid to adipic acid and ester.

FIG. 6 is a scheme showing the conversion of mucic acid to adipic acid ester via deoxydehydration/transfer hydrogenation process in one-pot.

FIG. 7A is the 1H NMR spectrum of compound 2.

FIG. 7B is the 1H NMR spectrum of compound 3.

FIG. 7C is the 1H NMR spectrum of compound 4.

FIG. 7D is the 1H NMR spectrum of compound 5.

FIG. 7E is the 1H NMR spectrum of compound 6.

FIG. 7F is the 1H NMR spectrum of compound 7.

FIG. 7G is the 1H NMR spectrum of compound 10.

### EXAMPLES

Non-limiting examples of the invention will be further described in greater detail by reference to specific Examples, which should not be construed as in any way limiting the scope of the invention.

### Example 1

### Materials

All starting materials are commercially available and were used as received, unless otherwise indicated. Mucic acid (98%), 3-pentanol (98%), TsOH (98%), 2-propanol (99.9%) were purchased from Merck; trans,trans-muconic acid (98%), 3-octanol (99%) and 5%Pt/C were purchased from Aldrich. Methyltrioxorhenium (MTO) (98%), Re₂O₇(99.99%) and Re₂(CO)₁₀ were purchased from Strem Chemical, USA; 1-butanol (99.5%) were purchased from BDH Laboratory Supplies, England. Other regents involved were from Sigma or Merck. ¹H and ¹³C NMR spectra were obtained using a Brucker AV-400 (400 MHz) spectrometer. Chemical shifts are reported in ppm with reference to tetramethylsilane with the solvent resonance as the internal standard.

### Example 2

### General Procedure for the Synthesis of Diethylmucate 6

A mixture of mucic acid (5.0 g), H₂SO₄ (1 ml), and ethanol (150.0 ml) was refluxed (80° C.) for 24 h under stirring. The reaction mixture was cooled to room temperature, and then stored at 3° C. for 1 day. The white precipitate was filtered out, washed with small amount of cold ethanol, and then vacuum dried at 50° C. overnight. The mother liquid was evaporated to dryness to give a brown solid. The solid obtained from the mother liquid was recrystallized in 10 ml ethanol and recovered by the procedure as described above. The total amount of the diethylmucate was 5.1 g (90.0% yield).

### Example 3

### General Procedure for the Deoxydehydration (DODH) of Mucic Acid

A mixture of mucic acid (1 mmol, 210 mg), methyltrioxorhenium (MTO) (0.05 mmol, 12 mg), TsOH (0.05 mmol, 12 mg), and 3-pentanol (20.0 ml) was refluxed (120° C.) in a 50 ml flask under flowing air or N₂. The mixture was initially a white suspension and then changed to a brown and transparent solution after 4 h. After 12 h, the reaction mixture was evaporated to dryness. The solid was recrystallized to get products. For kinetic study, 1 ml of reaction mixture was taken at certain time interval and dried for NMR analysis; known amount of mesitylene was added as an internal standard.

### Example 4

### General Procedure for the Deoxydehydration/Transfer-Hydrogenation of Mucic Acid to Adipic Acid Ester (One-Step)

A mixture of mucic acid (210 mg, 1 mmol), 5.0% Pt/C (10.0 mg), methyltrioxorhenium (MTO) (0.05 mmol, 12 mg), TsOH (0.05 mmol, 12 mg), and 3-pentanol (20.0 ml) was charged into a pressure flask. The reaction mixture was stirred at 200° C. for 36 hours (75% yield).

### Example 5

### General Procedure for the Deoxydehydration/Transfer-Hydrogenation of Mucic Acid to Adipic Acid Ester (Two-Step, One-Pot)

A mixture of mucic acid (210 mg, 1 mmol), methyltrioxorhenium (MTO) (0.05 mmol, 12 mg), TsOH (0.05 mmol, 12 mg), and 3-pentanol (20.0 ml) was charged into a pressure flask. The reaction mixture was stirred at 120° C. under flowing air for 12 hours. Then, 5.0% Pt/C (10.0 mg) was added into the flask. The flask was sealed and the reaction mixture was stirred at 200° C. for another 12 hours. The reaction mixture was then cooled down to room temperature. The catalyst was separated by filtration, the solvent was removed by evaporation, and adipic acid ester was obtained as a white liquid.

### Example 6

### Larger Scale synthesis of Adipic Acid Esters from Mucic Acid

A mixture of mucic acid (25.0 mmol, 5.25 g), MTO (1.25 mmol, 300 mg), TsOH (1.25 mmol, 215 mg), and 3-pentanol (250.0 mL) was charged into a pressure flask. The reaction mixture was stirred at 120° C. for 12 h. A water separator was used to remove the produced water. After that, 1.56 g of 5.0% Pt/C was added into the flask. The flask was sealed and the reaction mixture was stirred at 160° C. for another 12 h. The reaction mixture was then cooled down to room temperature. The catalysts were separated by filtration through Celite-545, the solvent was removed by evaporation, and the obtained adipic acid esters were purified by flash column chromatography (CHCl₃/MeOH 10:1) to give colorless liquid (6.84 g, 98% yield, dipentyl ester/ monopentyl ester 93:7).

### Example 7

### Hydrolysis of Adipic Acid Dipentyl Ester

Hydrolysis of adipic acid dipentyl ester: The separated adipic acid dipentyl ester (286.0 mg, 1 mmol) was refluxed for 12 h in an EtOH/H₂O solution of sodium hydroxide (0.133 molL⁻¹, 15.0 mL; EtOH/H₂O 1:2). After that, the reaction mixture was evaporated to dryness, and the obtained solid was dissolved in 10.0 mL deionized water. The pH value of the aqueous solution was adjusted to about 3.0 with 1M HCl. The solution was again evaporated to dryness, and the obtained solid was stirred in 10.0 mL methanol for 3 min. The mixture was then filtered through Celite-545, and the filtrate was evaporated to afford adipic acid as a white solid. The product was vacuum dried at 60° C. overnight, and adipic acid was obtained at 94% yield (136.8 mg).

### Example 8

### Initial Trial

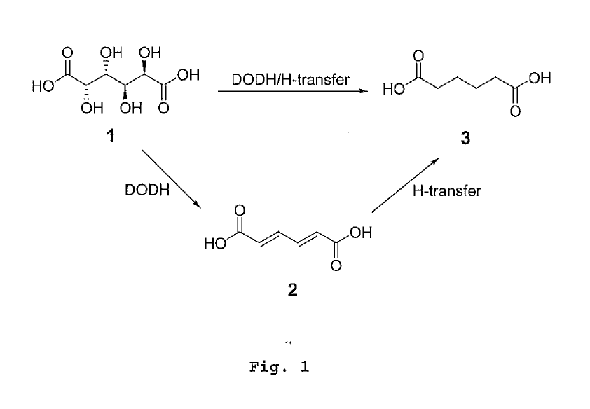

As shown in FIG. 1, Mucic acid 1 is a C₆ sugar acid that can be produced from galactose in large scale by the established method. The mucic acid was firstly tested under typical deoxydehydration (DODH) reaction condition with methyltrioxorhenium (MTO) catalyst. In the initial trial, mucic acid was heated in 3-octanol at 180° C. for 2 hours. The reaction went very slow and the selectivity was low. The majority of mucic acid remained as insoluble solid throughout the reaction.

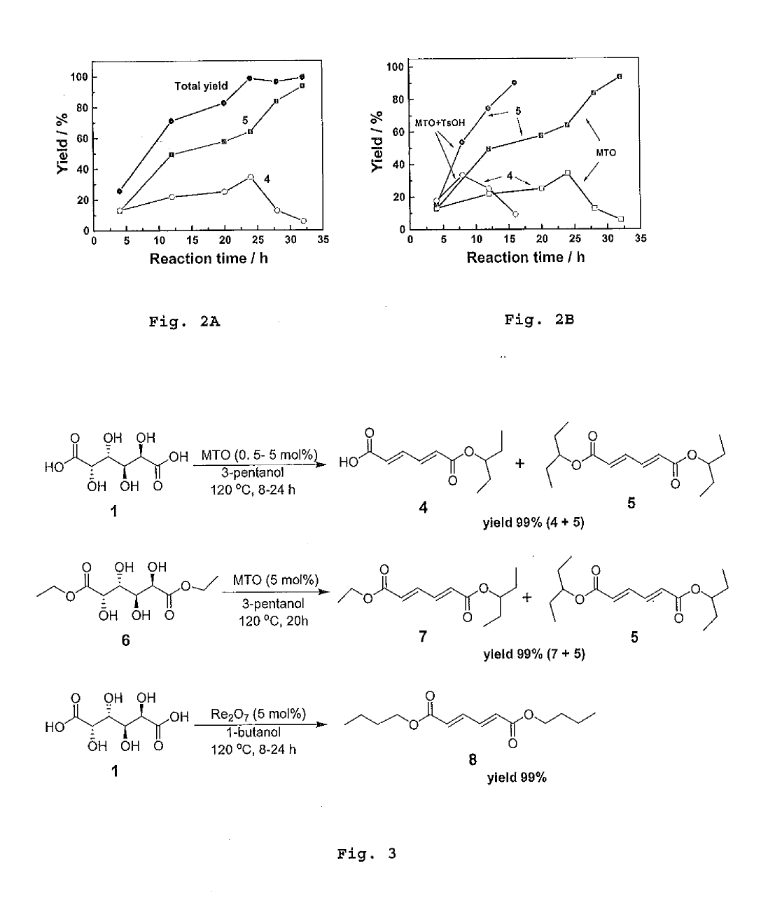

With 3-pentanol as the solvent, at 120° C., the reaction was still slow in the initial stage, probably due to low solubility of mucic acid, but with excellent selectivity. Kinetic study showed that mucic acid gradually converted to the conjugated double bond products in boiling 3-pentanol (120° C.) with 5 mol % MTO (FIG. 2A). It was found that the products were in the forms of monoester 4 and diester 5. The yield of monoester 4 was continuously increased and reached the maximum at 24 h, and then decreased. Meanwhile, the yield of diester 5 kept increasing even after 24 hours. It was clear that mucic acid was first converted to monoester 4 and then further converted to diester 5 (FIG. 3).

The full conversion to 4 and 5 was observed after 24 hours. Although the reaction temperature of this system is lower, the reaction rate is also slow as compared to other deoxydehydration (DODH) reactions of polyols. This could be due to the low solubility of mucic acid and/or the interference of carboxylic acid groups. In fact, higher temperature led to lower selectivity, while the reaction was sluggish at lower temperature (90° C.).

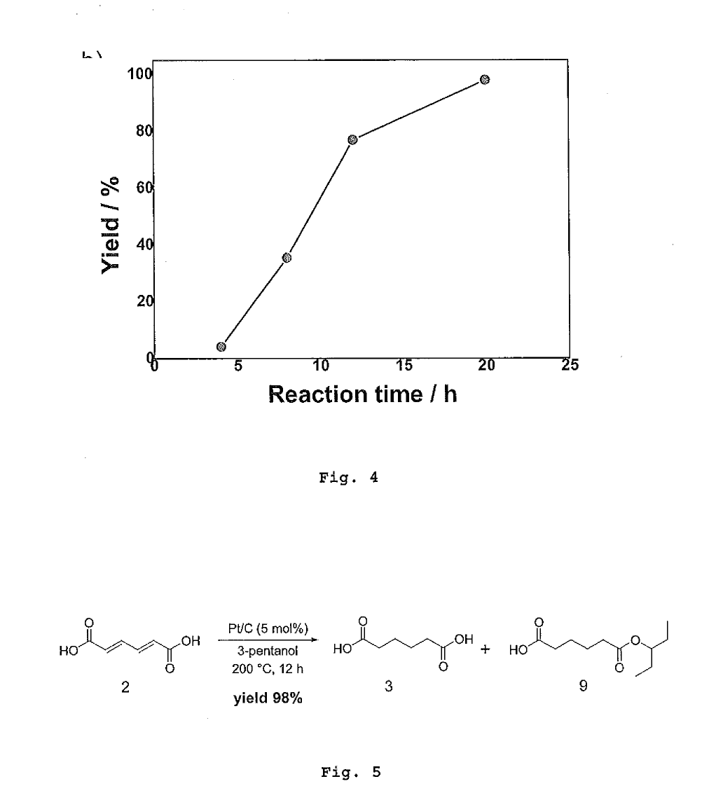

To understand more about this reaction, diethylmucate 6 (FIG. 3) was prepared according to reported method as the starting material for the deoxydehydration (DODH) reaction. Under the same reaction conditions, full conversion of diethylmucate 6 to muconate 5 (30%) and 7 (70%) were achieved at 20 hours (FIG. 4). The ester exchange reaction occurred and 65.0% of the ethyl groups were substituted by 3-pentanol. Despite the fact that diethylmucate has much better solubility than the free mucic acid in hot alcohols, the reaction of diethylmucate proceeded only slightly faster than that of mucic acid. This result may indicate that mucic acid was first subject to esterification before it underwent deoxydehydration (DODH) reaction. The reaction was generally conducted under air or N₂ flow (Entry 3, Table 1). In contrast, slower reaction was observed under the sealed system (Entry 4, Table 1) and much faster reaction was achieved when a water separator was employed (Entry 5, Table 1).

TABLE 1
Catalysts screening for the conversion of mucic acid to
muconates.^{a}

| | | Time | Conv. | Yield |
|---|---|---|---|---|
| Entry | Catalyst (mol %) | (h) | (%) | (%)b |
| 1 | CH3ReO3 (5) | 12 | 97.8 | 71.1 |
| 2 | CH3ReO3 (5) | 24 | 100.0 | 98.6 |
| 3 | CH3ReO3 (5) | 24c | 100.0 | 96.6 |
| 4 | CH3ReO3 (5) | 28d | 100.0 | 87.0 |
| 5 | CH3ReO3 (5) | 8e | 100.0 | 99.1 |
| 6 | CH3ReO3 (5), TsOH | 12 | 100.0 | 99.5 |
| | (5) | | | |
| 7 | CH3ReO3 (5), H2SO4 | 12 | 100.0 | 99.3 |
| | (5) | | | |
| 8 | CH3ReO3 (2.5), TsOH | 12 | 100.0 | 99.7 |
| | (5) | | | |
| 9 | CH3ReO3 (1.25), TsOH | 12 | 100.0 | 95.1 |
| | (2.5) | | | |
| 10 | CH3ReO3 (0.5), TsOH | 12 | 57.3 | 47.6 |
| | (2.5) | | | |
| 11 | CH3ReO3 (0.5), TsOH | 24 | 100.0 | 99.3 |
| | (2.5) | | | |
| 12 | Re2(CO)10 (5) | 12 | 0 | 0 |
| 13 | Re2O7 (5) | 4 | 100.0 | 87.3 |
| 14 | Re2O7 (5) | 8 | 100.0 | 95.4 |
| 15 | Re2O7 (5), TsOH (5) | 4 | 100.0 | 82.4 |
| 16 | Re2O7 (5), TsOH (5) | 8 | 100.0 | 99.8 |

^{a}Reaction conditions: mucic acid (1.0 mmol), 3-pentanol (20.0 ml), 120° C., flowing N₂.
bNMR yield of 4 + 5.
cFlowing air.
dClosed reaction system.
eWater separator was employed.

### Example 9

### Investigating the Effect of Brønsted Acids

In an attempt to accelerate the reaction, Brønsted acids were added as a co-catalyst to promote the esterification step to enhance the solubility of the starting material. As shown in FIG. 2B, the addition of para-toluene-sulfonic acid (TsOH) remarkably shortened the reaction time to reach the full conversion, while the products distribution and the trends of products formation remained the same. Sulfuric acid also showed the similar promoting effect (Entry 7, Table 1), In fact, the acid additives for the deoxydehydration (DODH) reaction would assist olefin extrusion by protonation of the rhenium diolate intermediate, while the extrusion of olefin from oxorhenium complex is the key step in deoxydehydration (DODH) reaction. With the aid of acid co-catalyst, the methyltrioxorhenium catalyst (MTO) loading can be reduced to as low as 0.5 mol % (Entries 8-11, Table 1).

### Example 10

### Investigating the Effect of Different Re Catalysts

Various Re catalysts were tested for the reaction. Re₂(CO)₁₀ is efficient for the deoxydehydration (DODH) of a variety of vicinal diols. However, Re₂(CO)₁₀ is inactive for the deoxydehydration (DODH) of mucic acid (Entry 12, Table 1), probably due to the poor tolerance of Re₂(CO)₁₀ to the carboxylic acid group. In contrast, high reaction rate was observed for the Re₂O₇ catalyzed deoxydehydration (DODH) reaction of mucic acid in 3-pentanol (Entries 13-14, Table 1). The reaction with Re₂O₇ catalyst is even faster than that catalyzed by methyltrioxorhenium (MTO) in combination with TsOH. Re₂O₇ is hydroscopic and can react easily with even the moisture to form HReO₄¹⁹, which may promote the esterification and olefin extrusion steps in the deoxydehydration (DODH) catalytic cycle. Addition of TsOH to the Re₂O₇ reaction system didn't further improve the reaction efficiency (Entries 15-16, Table 1).

### Example 11

### Investigating the Effect of Solvent

Though it can be operated at a higher temperature by using 3-octanol as the solvent, slower reaction was observed, As to the solvent of this reaction, 3-octanol is less active even though it can be operated at a higher temperature, probably due to the less polarity of 3-octanol (Table 2). 2-propanol is almost inactive, as the reaction was carried at lower temperature due to its low boiling point. The viability of using bio-derivable 1-butanol for this reaction has also been explored. It turned out that the reaction efficiency is similar to that of 3-pentanol, almost quantitative conversion of mucic acid to muconate was achieved in 12 hours with Re₂O₇ as catalyst (Entry 3, Table 2). In 1-butanol system, only dibutyl-muconate 8 (FIG. 2) was observed, probably due to the less steric effect of primary alcohol.

TABLE 2
Deoxydehydration (DODH) reaction of mucic acid in
different alcohol solvents.

| | | T | t | | |
|---|---|---|---|---|---|
| Entry | Solvent | (° C.) | (h) | Conv. [%] | Yield [%] |
| 1 | 3-octanol | 180 | 20.0 | 100.0 | 67.0 |
| 2 | 3-pentanol | 120 | 12.0 | 97.8 | 71.1 |
| | | | 24.0 | 100.0 | 98.6 |
| 3 | 1-butanol | 120 | 24.0 | 78.9 | 68.7 |
| | | | 12.0^{b} | 100.0 | 99.7^{c} |
| 4 | 2-propanol | 90 | 24.0 | 9.6 | — |

^{a} Reaction conditions: mucic acid (1.0 mmol), methyltrioxorhenium (MTO) (0.05 mmol, 5.0 mol %), 3-pentanol (20.0 ml). Yield and conversion were determined by ¹H NMR with internal standard.
^{b}Re₂O₇ (0.05 mmol, 5.0 mol %) was used as catalyst, n-butyl muconate 8 was produced.

### Example 12

### Hydrogen Transfer Reaction

As the high yield of muconates from mucic acid was achieved, subsequently, hydrogen transfer reaction was demonstrated for the conversion of muconic acid or muconate to adipic acid or ester (FIG. 5) Firstly, muconic acid 2 could be quantitatively converted to adipic acid 3 or monoester 9 in 3-pentanol at 200° C. in 12 to 24 hours. Similar results were achieved when different catalysts (Ru/C, Pd/C or Pt/C) were used. Encouraged by this result, crude muconates that obtained from deoxydehydration (DODH) reaction were directly used as feedstock for the hydrogen transfer reaction. Remarkably, the muconates obtained from mucic acid via deoxydehydration (DODH) reaction were fully converted to adipic acid esters 9 and 10 (Table 3). The reaction is highly selective and no other by-product was observed.

TABLE 3
Catalyst screening for transfer hydrogenation reaction.^{a}

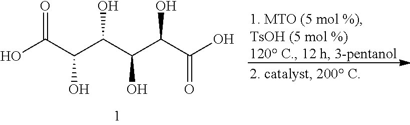 [SMILES: O=C(O)[C@@H](O)[C@H](O)[C@H](O)[C@@H](O)C(=O)O]

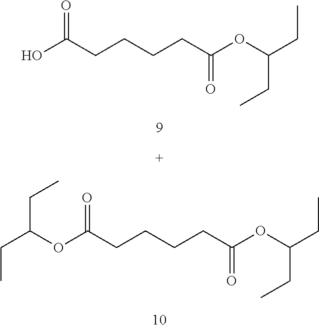 [SMILES: CCC(CC)OC(=O)CCCCC(=O)O ; CCC(CC)OC(=O)CCCCC(=O)OC(CC)CC]

| Entry | Catal. | t (h) | Conv. (%) | Yield (%)^{b} |
|---|---|---|---|---|
| 1 | 5%Ru/C | 24 | 100.0 | 99.5 |
| 2 | | 12 | 100.0 | 86.3 |
| 3 | | 6 | 82.7 | 81.7 |
| 4 | 5%Pd/C | 24 | 60.4 | 38.5 |
| 5 | | 12 | 54.4 | 29.0 |
| 6 | | 6 | 45.9 | 23.4 |
| 7 | 5%Pt/C | 24 | 100.0 | 99.5 |
| 8 | | 12 | 100.0 | 99.3 |
| 9 | | 6 | 78.2 | 76.3 |

^{a}Reaction conditions:
1. mucic acid (1 mmol); 3-pentanol (20.0 ml);
2. catalyst (5 mol %).
^{b}NMR yield of 9 and 10.

### Example 13

### One Pot Reaction

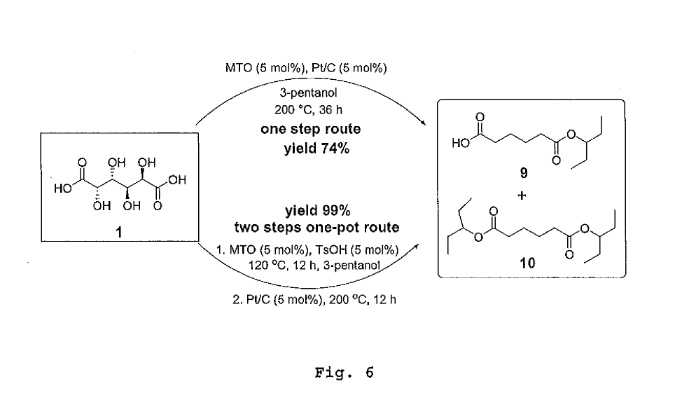

Since both deoxydehydration (DODH) and hydrogen transfer reaction could be conducted in 3-pentanol, the one-pot reaction for the conversion of mucic acid to adipic acid or ester was tested (FIG. 6). In a closed reaction system with methyltrioxorhenium (MTO) and Pt/C catalysts, mucic acid was converted to adipic acid ester in 75% yield at 200° C. in 48 hours. The moderate efficiency is due to the low reaction rate for the deoxydehydration (DODH) step in the closed system, as mentioned above. As the reaction was carried in the manner, first conducted at 120° C. under air flow for 12 hours and then sealed the system to elevate the temperature to 200° C. for further reaction for another 12 hours, the one-pot reaction efficiency was dramatically increased and 99% of adipic acid ester was achieved directly from mucic acid in 24 hours. As mucic acid can be obtained from renewable galactose in large scale by the already established procedure, the method developed here provided a highly efficient synthetic protocol for the production of renewable adipic acid.

### Example 12

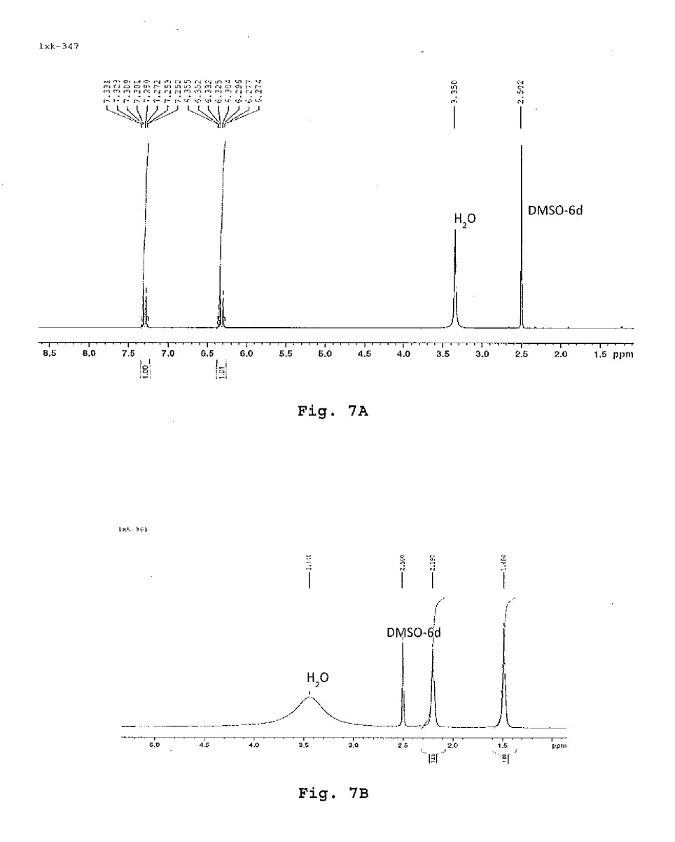

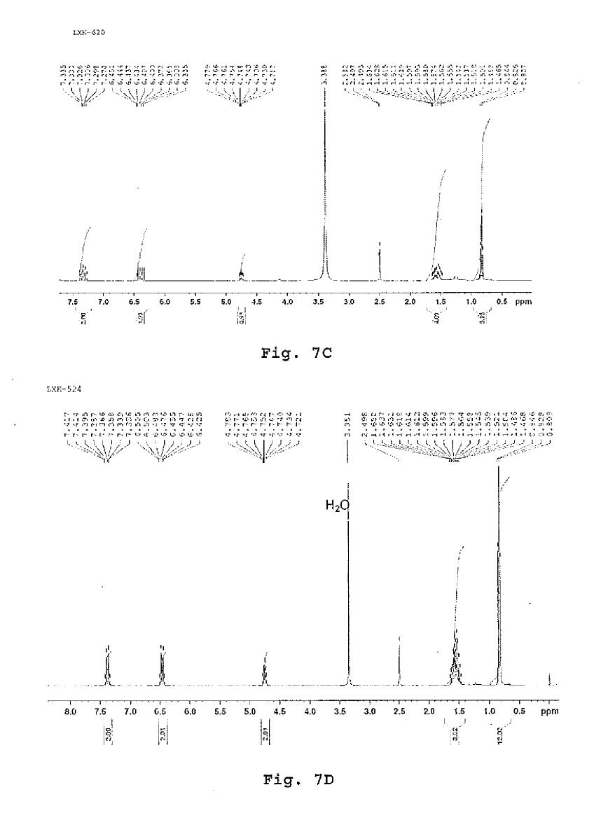

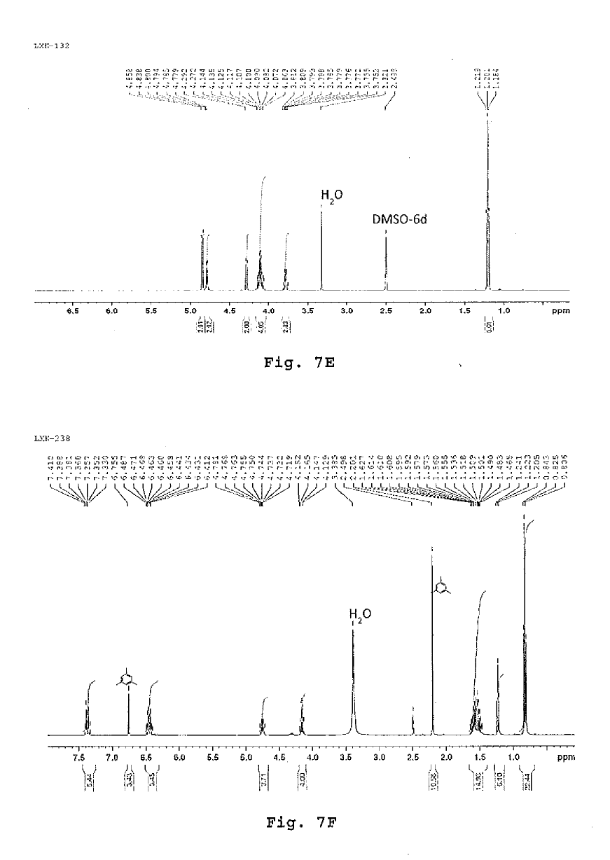

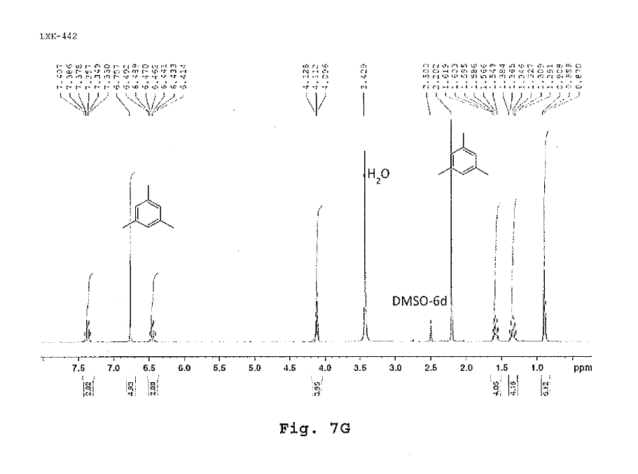

### Compound Characterization

| Compound | NMR characterization | NMR spectrum |
|---|---|---|
| 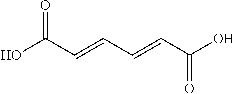 | 1H NMR (400 MHz, DMSO- 6d), δ = 7.33-7.25 (m, 2H); δ = 6.36-6.27 (m, 2H). | FIG. 7A |
| 2 | | |
| 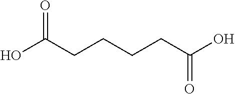 | ¹H NMR (400 MHz, DMSO- 6d), δ = 2.20 (s, 4H); δ = 1.48 (s, 4H). | FIG. 7B |
| 3 | | |
| 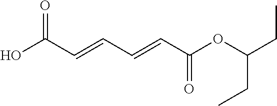 | ¹H NMR (400 MHz, DMSO- 6d) , δ = 7.41-7.27 (m, 2H); δ = 6.49-6.29 (m, 2H); δ = 4.78-4.72 (m, 1H); δ = 1.63-1.46 (m, 4H); δ = 0.84, 0.83, | FIG. 7C |
| 4 | 0.81 (t, J = 7.4 Hz, | |
| | 6H, CH₃). | |
| 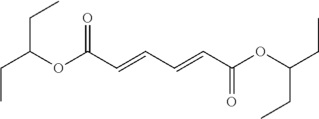 [SMILES: CCC(CC)OC(=O)/C=C/C=C/C(=O)OC(CC)CC] | ¹H NMR (400 MHz, DMSO- 6d), δ = 7.35 (q, 2H); δ = 6.45 (q, 2H); δ = 4.78-4.72 (m, 2H) ; δ = 1.63-1.46 (m, 8H); δ = 0.84, 0.83, 0.81 (t, J = 7.4 Hz, 12H, CH₃). | FIG. 7D |
| 5 | | |
| 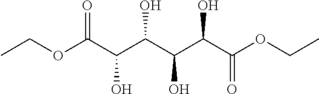 [SMILES: CCOC(=O)[C@@H](O)[C@H](O)[C@H](O)[C@@H](O)C(=O)OCC] | ¹H NMR (400 MHz, DMSO- 6d) , δ = 4.85 (d, J = 8.0 Hz, 2H, CH—OH); 4.79 (m, 2H, OH); 4.28 (d, J = 8.0 Hz, 2H, CH—OH); 4.11 (m, 4H, | FIG. 7E |
| 6 | CH₂); 3.78 (m, 2H, | |
| | OH); 1.20 (t, J = | |
| | 8.0 Hz, 6H, CH₃). | |
| 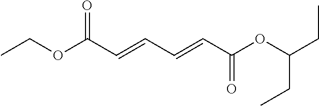 [SMILES: CCOC(=O)/C=C/C=C/C(=O)OC(CC)CC] | ¹H NMR (400 MHz, DMSO- 6d), δ = 7.37 (m, 2H); 6.45 (m, 2H); 4.75 (m, 1H, CH); 4.16 (m, 2H, CH₂); 1.55 (m, 4H, CH₂); 1.22 (t, J = | FIG. 7F |
| 7 | 7.2 Hz, 3H, CH₃); 0.83 (t, | |
| | J = 7.4 Hz, 6H, CH₃). | |
| 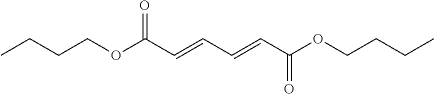 [SMILES: CCCCOC(=O)/C=C/C=C/C(=O)OCCCC] | ¹H NMR (400 MHz, DMSO- 6d), δ = 7.37 (m, 2H); 6.45 (m, 2H); 4.11 (t, J = 6.4 Hz, 4H); 1.58 (m, 4H, CH₂); 1.34 (m, 4H, CH₂); 0.89 (t, J = | FIG. 7G |
| 8 | 8.0 Hz, 6H, CH₃). | |

### Example 14

### Summary

In conclusion, the highly efficient synthetic protocol for the conversion of mucic acid to muconic acid, and then adipic acid through oxorhenium complex catalyzed deoxydehydration (DODH) Pt/C catalyzed hydrogen transfer sequence was demonstrated. Almost quantitative yields were achieved from mucic acid to muconic acid and The result presented here not only demonstrated a high efficient, simple and green protocol for the production of renewable adipic acid from sugar acid. It indicates the huge potential of formation of various industrial chemicals from various sugar acids.

### Applications

The disclosed method is useful in synthesizing an ester of a saturated carboxylic acid from a polyhdroxycarboxylic acid.

The disclosed method may be used to convert mucic acid to adipic acid, which is used commonly as a monomer precursor for the production a variety of polymers including nylon and polyurethane. Adipic acid may also be used in medicine, such as in controlled-release formulation matrix tablets to obtain pH-independent release of both weakly basic and weakly acidic drugs. In addition, small but significant amounts of adipic acid may be used in food as a flavorant or gelling aid. The disclosed method may therefore be useful in the industrial-scale production of adipic acid for the above applications.

The disclosed method may simplify the synthetic process of saturated carboxylic acids such as adipic acids from polyhdroxycarboxylic acids such as mucic acid, as the reaction conditions are milder and more time- and cost-efficient compared to conventional methods.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for synthesizing an ester of a saturated carboxylic acid, the method comprising:
(a) subjecting a polyhydroxycarboxylic acid to a deoxydehydration catalyst to remove hydroxyl groups; and
(b) performing a hydrogen transfer reaction to form the ester of a saturated polycarboxylic acid.

2. The method according to claim 1, wherein the hydrogen transfer reaction in operation (b) is performed in the presence of a hydrogen transfer catalyst.

3. The method according to claim 1, wherein operations (a) and (b) are performed in a single reaction vessel.

4. The method according to claim 3, wherein operations (a) and (b) are performed concurrently or consecutively.

5. The method according to claim 4, wherein the polyhydroxycarboxylic acid is contacted with the dehydration catalyst in the presence of the hydrogen transfer catalyst.

6. (canceled)

7. The method according to claim 4, wherein the deoxydehydration catalyst produces at least one intermediate compound, wherein the at least one intermediate compound produced is an ester of an unsaturated polycarboxylic acid after operation (a).

8. (canceled)

9. The method according to claim 7, wherein the ester of an unsaturated polycarboxylic acid is further subjected to operation (b) to produce the ester of a saturated polycarboxylic acid.

10. The method according to claim 1 wherein the ester of an unsaturated polycarboxylic acid comprises esters of muconic acid selected from the group consisting of propyl muconate, dipropyl muconate, butyl muconate, dibutyl muconate, pentyl muconate, dipentyl muconate, octyl muconate, dioctyl muconate and any mixture thereof.

11. (canceled)

12. (canceled)

13. The method according to claim 1, wherein the ester of a saturated polycarboxylic acid is an ester of adipic acid selected from the group consisting of adipic acid monopropyl ester, adipic acid dipropyl ester, adipic acid monobutyl ester, adipic acid dibutyl ester, adipic acid monopentyl ester, adipic acid dipentyl ester, adipic acid monooctyl ester, adipic acid dioctyl ester and any mixture thereof.

14. (canceled)

15. (canceled)

16. The method according to claim 1, wherein the polyhydroxycarboxylic acid is mucic acid.

17. The method according to claim 1, wherein the deoxydehydration catalyst in operation (a) is a rhenium catalyst or a rhenium catalyst with a co-catalyst, wherein the rhenium catalyst is rhenium acid, methyltrioxorhenium or rhenium(III) oxide or rhenium acid and, wherein the co-catalyst is a BrØnsted acid or a proton type liquid or solid acid or para-toluene sulfonic acid or sulfuric acid.

18-21. (canceled)

22. The method according to claim 1, wherein the hydrogen transfer catalyst in operation (b) is a metal-on-carbon catalyst containing a metal, wherein the metal is selected from the group consisting of platinum, palladium, ruthenium and any mixture thereof, or the hydrogen transfer catalyst is selected from the group consisting of Ru/C, Pd/C, Pt/C and any mixture thereof.

23. (canceled)

24. (canceled)

25. The method according to claim 17, wherein the hydrogen transfer catalyst comprises up to 5 mol % of the reaction mixture.

26. The method according to claim 4, wherein the method further comprises the use of an alcohol solvent.

27. (canceled)

28. The method according to claim 4, wherein the method is performed at a temperature in the range of 120° C. to 200° C. or for a duration in the range of 24 hours to 36 hours.

29. (canceled)

30. The method according to claim 7, wherein the method further comprises the use of an alcohol solvent in operation (a) and operation (b), wherein the alcohol solvent in operation (a) is selected from the group consisting of propanol, butanol, pentanol, hexanol, heptanol, octanol and any mixture thereof, or is selected from the group of 2-propanol, 1-butanol, 3-pentanol, 3-octanol and any mixture thereof, and wherein the alcohol solvent in operation (b) is 3-pentanol.

31-33. (canceled)

34. The method according to claim 7, wherein operation (a) is performed at a temperature in the range of 90° C. to 180° C. and operation (b) is performed at a temperature in the range of 120° C. to 200° C., or operation (a) is performed for a duration of 4 hours to 24 hours and operation (b) is performed for a duration of 6 hours to 24 hours.

35. (canceled)

36. An ester of adipic acid synthesized according to a method for synthesizing an ester of a saturated carboxylic acid, the method comprising:
(a) subjecting a polyhydroxycarboxylic acid to a deoxydehydration catalyst to remove hydroxyl groups; and
(b) performing a hydrogen transfer reaction to form the ester of a saturated polycarboxylic acid.

37. A method for synthesizing an ester of adipic acid, the method comprising the operation of subjecting mucic acid to a deoxydehydration catalyst in the presence of a hydrogen transfer catalyst to form the ester of adipic acid, or the operations of:
(a) subjecting mucic acid to a dehydration catalyst to form an ester of muconic acid; and
(b) performing a hydrogen transfer reaction on the ester of muconic acid to form the ester of adipic acid.

38. (canceled)

39. A method for synthesizing a saturated carboxylic acid, the method comprising:
(a) subjecting a polyhydroxycarboxylic acid to a deoxydehydration catalyst to remove hydroxyl groups;
(b) performing a hydrogen transfer reaction to form the ester of a saturated polycarboxylic acid; and
(c) hydrolysing the ester to form the saturated carboxylic acid.
